(19) United States
(12) Patent Application Publication
(10) Pub. No.: US 2022/0139519 A1
(43) Pub. Date: May 5, 2022

(54) SMART PILL ORGANIZER TO FACILITATE PATIENT ADHERENCE BY REMOTELY MONITORING MEDICINE USAGE IN A HOME

(71) Applicant: Robert Joseph Schilling, Potsdam, NY (US)

(72) Inventor: Robert Joseph Schilling, Potsdam, NY (US)

(21) Appl. No.: 17/084,011

(22) Filed: Oct. 29, 2020

(51) Int. Cl. G16H 20/13; G16H 40/67; G16H 10/60; G16H 40/40; G06Q 10/10; G16H 15/00; G16H 40/20; G06Q 30/00; G06F 8/65; A61J 7/00; A61J 7/04

(52) U.S. Cl. CPC G16H 20/13; G16H 40/67; G16H 10/60; G16H 40/40; G06Q 10/107; G06Q 10/109; A61J 2205/70; G16H 40/20; G06Q 30/016; G06F 8/65; A61J 7/0069; A61J 7/04; A61J 2200/30; G16H 15/00

(57) ABSTRACT

A smart pill organizer or pillbox facilitates patient adherence by remotely monitoring medicine usage in a home. A patient web site is accessible locally on the Wi-Fi network by the patient or remotely over the Internet by a guardian who can be a relative, health care provider, or service. Remote pillbox access is also available through a set of email and text commands. Dose types include pillbox doses in pill tray bins, external doses in their original medicine containers, and remote doses taken away from home. Dose frequencies include monthly, weekly, daily, and multiple doses per day. Local and remote dose reminders are provided. Pillbox doses are recorded when the pillbox bin opens, while external and remote doses are recorded with replies to email and text reminders. Real time numerical adherence measures ranging from 0 to 100% are provided for dose quantity, dose timing, dose spacing, and overall adherence.

### BACKGROUND

Since the 1970s medical research has focused on the problem of patient adherence (also called patient compliance) to prescribed medicine regimens. Lack of patient adherence is a complex problem with many causes that is estimated to generate significant medical costs in both hospital and nursing home admissions. Less than complete patient adherence has been observed to occur in many patients. Lack of adherence can be intentional or unintentional, and includes such things as incorrect dosage, taking doses at the wrong time, forgetting to take one or more doses, and ceasing to take the medication. It is not uncommon, for example, for patients to reduce the dosage in order to limit side effects or to make expensive medication last longer.

Accurate and reliable measurement of patient adherence is a challenging ongoing problem that has no widely accepted solution. Direct traditional measures of adherence involve measurement of body fluid (blood or urine) to detect the concentration of the drug or its metabolite or marker. Direct observation is sometimes used but this is applicable mainly for hospitalized or nursing home patients. Direct methods are considered the most accurate, but they are both invasive and costly. Several indirect measurement methods have been used, although they tend to be less accurate. These include self-reporting methods such as patient interviews and the use of diaries. Periodic pill counts at doctor's offices also have been used. Less cumbersome for the patient is the use of prescription refill dates, but here the adherence results are delayed and less precise. Outcome measures also have been used, but sometimes it can be difficult to directly correlate outcomes with patient adherence.

The use of customized electronic devices to measure patient adherence tends to be more accurate, but it also can be more expensive. In addition to measuring patient adherence these devices also attempt to improve adherence by providing reminders to the patient that it is time to take medication. Prior art includes a number of devices that have been developed that extend and enhance the widely-used basic pill organizer. Most devices are restricted to tablets and capsules that fit within the bins of a pill organizer, pill dispenser or pillbox. There are several different styles of medication that are not easily accommodated by pill tray bins such as inhalers, injections, eye drops, liquid medication that may or may not have to be refrigerated, and medicine in blister packs. Younger patients who are not retired often have to take medicine at work as well as at home. Medication is taken monthly, weekly, daily, and multiple times per day. To accommodate this wide variety of circumstances, a plurality of dose types should be supported where a dose is any collection of medications that can be taken at the same time. To support multiple dose types, multiple dose reminder types and multiple of means of recording doses are required.

Prior art also includes devices that provide a patient web site, typically on a centralized server, that communicates with the pillbox. Patient web sites provide adherence results and they also can be used to remotely configure the pillbox. Given current technology, it is possible to host the patient web site locally in the pillbox itself. This has the advantage that the patient can access it through a local Wi-Fi network even when an Internet connection not present. It is increasingly the case that individuals make use of cell phones for email, and text messaging. Although a patient web site is an effective way to communicate with a pillbox, there is an equally effective alternative approach that uses short email and text commands. Secure commands can be sent directly to the pillbox from a restricted set of users including the patient and a guardian who monitors the progress of the patient in adhering to the medicine regimen. These commands can both request adherence results and configure the pillbox to accommodate the needs and desires of the patient. Email and text commands have the advantage that they can be sent using both a cell phone network and an Internet connection.

With prior art there does not appear to be a widely accepted standard way to report patient adherence results. The smart pill organizer provides real time numerical measures of patient adherence that include dose quantity, dose timing, dose spacing and overall adherence. The measures range from 0% to 100% where 100% represents perfect adherence and 0% corresponds to a complete lack of adherence. For example, if the patient takes only half of the scheduled doses, but the doses that are taken are taken at their scheduled times, then the dose quantity adherence is 50% and the dose timing adherence is 100%.

### SUMMARY

This simplified summary provides a basic understanding of some aspects of an embodiment of the smart pill organizer, more simply referred to as the pillbox. A more comprehensive overview of the essential features and scope of the pillbox can be found in the detailed description section.

The pillbox is a smart pill organizer designed to facilitate patient adherence by recording and remotely monitoring medicine usage in a home. It is designed for use by two individuals, a patient who takes the medication, and a guardian who oversees the patient's progress in following the prescribed medicine regimen. The guardian might be a family member, health care provider, or service that remotely monitors patient adherence using a digital device such as a PC, lap top, tablet or cell phone.

The embodiment of the pillbox consists of two instrumented weekly pill trays mounted on an enclosure that includes custom electronics designed to issue patient reminders, monitor patient adherence, host a patient web site, and facilitate communication between the patient and the guardian using email and text messaging. An alternative embodiment in a larger enclosure can include four or more pill trays.

The front of the pillbox features three LEDs. The blinking red LEDs provide reminders that it is time for a dose, a dose is late, or the pillbox is empty, while the solid green LED indicates that the pillbox is connected to the Internet. There are also bright white LEDs inside each pill tray bin that blink to show the patient which bin to remove a dose from. Here a dose is any assortment of medications that can be taken at the same time. Pillbox doses are recorded by micro switches inside the bins that detect that a lid has been opened. The back of the pillbox includes two pushbuttons. When the pillbox status button is pushed the pillbox plays a voice message that announces the day, date, time, dose schedule, an adherence summary, and directions for connecting to the patient web site. The recessed password reset button restores the web site usernames and passwords of the patient and guardian to their default values. The back of the pillbox also contains an external memory stick (a USB flash drive) that is used to customize the pillbox for use by the patient. It also includes a wired network connector for wired Internet operation and a power connector. The pillbox is powered with a 5V DC wall transformer (12 W) similar to that used to charge cell phones.

Access to the patient web site is available locally on the patient's Wi-Fi network by entering the pillbox private IP address into a web browser. The guardian can access the patient web site remotely over the Internet using the public IP address of the patient's Wi-Fi network once the patient's router has been configured for port forwarding. A second way to remotely access the patient pillbox is through the use of a comprehensive set of email and text commands that duplicate and extend the functionality available on the patient web site. Using the patient web site or the email and text commands, the patient and the guardian can access patient dosage history and adherence data. They also can configure the pillbox options to customize it to fit the needs and desires of the patient. Email and text commands can be sent to more than one pillbox on the same Wi-Fi network, and an administrator can use an email command to remotely update the pillbox software to a new version. Unlike a patient web site, email and text commands can be sent using both a cell phone network and an Internet connection.

The pillbox supports five types of reminders and three types of doses. Local reminders include light (blinking LEDs), sound (audio beeping), and descriptive voice messages while remote reminders include descriptive email and text messages. When needed, dose reminders are placed on hold until sufficient time has elapsed to satisfy an adjustable minimum dose spacing requirement. The three dose types are pillbox doses, external doses, and remote doses. Pillbox doses are stored in pillbox bins and are taken at home. External doses also are taken at home but they do not fit in a pillbox bins so they are instead stored in their original medicine containers. Examples of external doses include inhalers, eye drops, injections, liquid medication, and pills in blister packs. Remote doses are doses taken at work or away from home and stored in separate medicine containers. While a pillbox dose is recorded when the bin containing the dose is opened, external and remote doses are recorded when the patient replies to an email or text reminder. Remote doses also can be recorded using email and text commands or the patient web site. Doses can be of one type on some days of the week and another type on other days of the week. Medication can be scheduled monthly, weekly, daily, or multiple times per day.

Unlike basic pill organizers, the pillbox provides the user with numerical measures of patient adherence including dose quantity, dose timing, dose spacing, and overall adherence. Adherence measures are computed in real time with patient adherence updated every time a dose is taken. Numerical adherence measures range from 0% to 100% where 100% correspond to perfect adherence. Weekly summaries of patient adherence and monthly dosage history summaries are emailed to the guardian to help monitor the progress of the patient. A patient adherence summary is available at any time using the pillbox status pushbutton.

The pillbox supports a feature called direct voice messaging. The patient and the guardian can schedule brief messages using the patient web site or email and text commands. For example, a message might remind the patient of a doctor's appointment or perhaps include words of encouragement from the guardian. The messages are direct in the sense that with normal pillbox operation the patient is guaranteed to hear the message because it is played on the scheduled delivery date when the patient opens a bin to take a dose.

The role of the guardian is to oversee the physical well being of the patient. In addition to monitoring patient adherence, the guardian also can monitor the state of the patient's environment by remotely measuring the temperature and the states of multiple external switches placed throughout the patient's home. This allows the guardian to detect if the temperature falls below a lower limit due to a problem with a furnace or rises above an upper limit due to a malfunctioning air conditioning system. In this event a remote alert is issued. The temperature limits are adjustable using the patient web site or email and text commands.

The email and text commands and the patient web site allow the user to create and edit a patient medication list that can be taken to doctor's appointments. When the medicine list is created or updated, it automatically generates descriptive dose reminders and a separate form that can be used to refill the pillbox.

### BRIEF DESCRIPTION OF THE DRAWINGS AND FIGURES

FIG. 1 Perspective view of the front and top of the pillbox

FIG. 2A Drawing showing cutouts in the front, bottom, and back of the pillbox enclosure

FIG. 2B Drawing showing cutouts in the cover of the pillbox enclosure

FIG. 3 Close-up perspective view of open bins showing mechanical micro switches, bin LEDs, and bin brackets.

FIG. 4 Disassembled view of pill tray cover, pill tray printed circuit board (PCB), pill tray gasket, and pill tray brackets.

FIG. 5A Schematic diagram of pill tray PCB

FIG. 5B Parts layout of pill tray PCB

FIG. 6 Perspective view of populated measurement and control PCB

FIG. 7A Schematic diagram of measurement and control PCB

FIG. 7B Parts layout of measurement and control PCB

Each of the individual parts shown in the figures and drawings has a unique part ID number with the list of part ID numbers summarized in Table 1.

TABLE 1
Part ID Numbers for Smart Pill Organizer Drawings and Figures

| Part ID | Fig. | |
|---|---|---|
| Number | Number | Description |
| 11 | 1 | Pill tray |
| 12 | 1 | Bin lid |
| 13 | 1 | Status lights |
| 14 | 2A | Status lights slot |
| 15 | 2A | Speaker mount holes (4x) |
| 16 | 2A | Ventilation holes (2x) |
| 17 | 2A | PCB mount holes (4x) |
| 18 | 2A | External memory, network sockets slot |
| 19 | 2A | Pillbox status pushbutton hole |
| 20 | 2A | Power connector hole |
| 21 | 2A | Password reset pushbutton hole |
| 22 | 2B | Pill tray mount holes (4x) |
| 23 | 2B | Ribbon cable slots (2x) |
| 24 | 3 | Micro switch (7x) |
| 25 | 3 | Bin light (7x) |
| 26 | 3 | Bracket mounting screw (7x) |
| 27 | 4 | Pill tray PCB |
| 28 | 4 | Pill tray cover |
| 29 | 4 | Bin bracket (7x) |
| 30 | 4 | Pill tray gasket |
| 31 | 6 | Sound actuator |
| 32 | 6 | Temperature sensor |
| 33 | 6 | Ribbon cable connectors (2x) |
| 34 | 6 | Processor board |
| 35 | 6 | Password reset pushbutton |
| 36 | 6 | Pillbox status pushbutton |
| 37 | 6 | Audio amplifier |
| 38 | 6 | External switch connector |
| 39 | 6 | Real time clock |

### DETAILED DESCRIPTION

This is a description of an embodiment of the smart pill organizer. For convenience the smart pill organizer is often referred to more simply as the “pillbox”. The pillbox is designed to facilitate patient adherence by recording and remotely monitoring medicine usage in a home. It is further designed for use by a pair of individuals, a patient who takes the medication, and a guardian who oversees the patient's progress in following the prescribed medicine regimen. One example of a patient might be an elderly parent living alone with the guardian being a son or daughter living at another location. More generally, the guardian could be a health care provider or a service. Remote access to the pillbox by the guardian is available using digital devices including PCs, lap tops, tablets and cell phones.

The smart pill organizer has been under development for some time. As experience with using the pillbox accumulates, new user features are added, particularly those that can be accommodated with updates to the underlying software. The embodiment described in this document uses a Raspberry Pi 3B+ processor board and software written in C, PCP, and HTML. Alternative embodiments may employ more advanced processing units and different programming languages. This description starts with a characterization of the hardware. This is followed by a description of user features for both the patient and the guardian. Next the implementation of the patient web site is presented. Finally an overview of the pillbox software is provided.

Pillbox Hardware

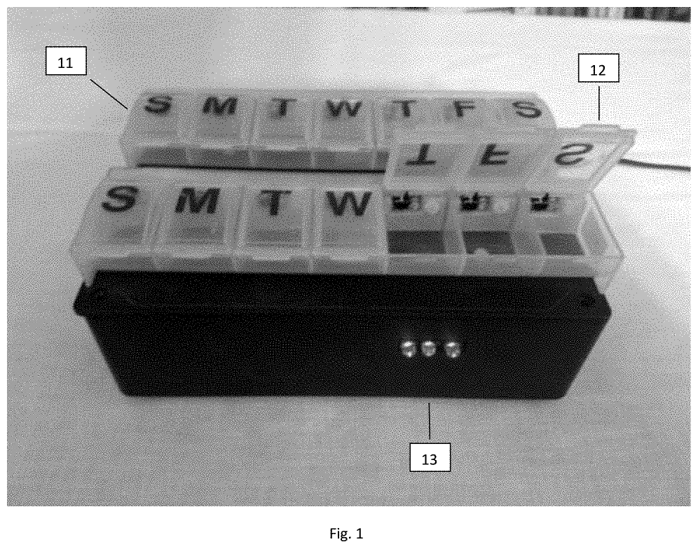

FIG. 1 shows a perspective view of the top and the front of the pillbox. The embodiment includes two weekly pill trays 11, but additional trays can be accommodated using a larger enclosure. The pill trays are plain plastic pill trays labeled with the days of the week. The pill tray bins are large enough to hold a generous assortment of tablets and capsules and they are curved at the bottom to accommodate the removal of pills without tipping the pill tray. Each pill tray is modified with custom electronics, as described below, to detect the open and closed positions of the bin lids 12 which are not at present lockable. Before the pillbox is first used, the pill trays must be filled with medicine and all of the lids closed. Each bin contains a dose where a dose is taken to mean any assortment of medications that can be taken at the same time. These are called pillbox doses because they are stored in the pillbox bins. Other types of doses are supported including external doses that do not fit in pillbox bins and are instead stored in their original medicine containers and remote doses that can be taken at work or away from home and stored in separate medicine containers. When it is time to take a pillbox dose, the patient opens the lid and removes the medication. The empty bin is then left open until it is time to refill the pillbox and repeat the process. Pillbox doses are recorded when a bin lid is opened. The patient is instructed with a voice message to leave the empty bin open until it is time to refill the pillbox.

Shown in FIG. 1 are three light emitting diodes (LEDs) that serve as status lights 13 on the front of the pillbox. The two on the ends are red LEDs that blink alternately when it is time to take a dose or a dose is late. They blink simultaneously when the bin is already open or the pillbox is empty. The status LED in the center is a green LED that activates to indicate that the pillbox is connected to the Internet. There are also bright white bin LEDs 25 that blink inside each bin to inform the patient which bin to remove the dose from. This way the patient does not have to remember the day of the week or the time of day when taking a dose.

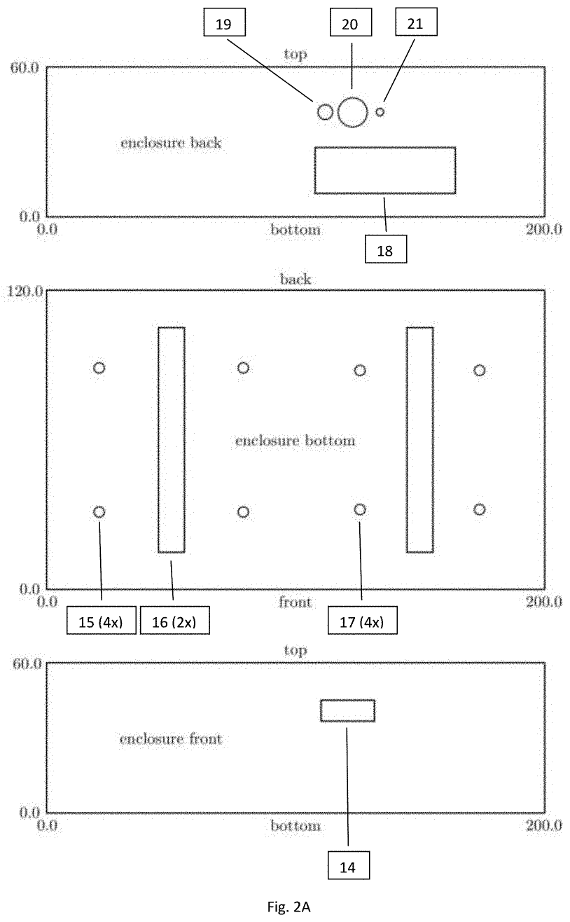

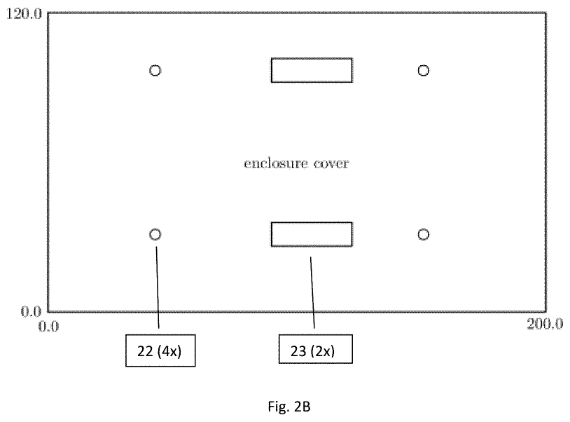

The electronics of the pillbox are housed in a plain ABS plastic enclosure. This enclosure is modified with custom cutouts to accommodate the user interface to the electronics. FIG. 2A shows the cutout patterns of the front, bottom, and back of the enclosure, while FIG. 2B shows the cutout pattern of the enclosure cover. The cutout in the front face is a horizontal slot 14 for the three status lights. The bottom cutouts include four mounting holes 15 for a speaker, two vertical slots 16 for ventilation, and four mounting holes 17 for a printed circuit board (PCB) assembly. The back of the enclosure has several cutouts. Slot 18 accommodates an external memory stick (a USB flash drive) that includes a plain text file wifi.txt that contains information used to customize the pillbox for use by the patient. Most of the information stored in wifi.txt also can be supplied by the patient or the guardian using email and text commands or the patient web site. The external memory stick also includes a second plain text file called medication_list.txt that lists the patient's medications. Like the file wifi.txt, this information also can be supplied or edited using the patient web site or email and text commands. Using the information in the file medication_list.txt, descriptive dose reminders are created and a pillbox refill form is generated. This form provides detailed directions for refilling the pillbox, and it also can be used for visits to a doctor's office either in hard copy form or on the patient's cell phone. Slot 18 also includes a network connector that allows the pillbox to operate using either a wired or a wireless Internet connection.

The next cutout on the back of the enclosure is a circular hole 19 for the pillbox status pushbutton. When this button is pushed it plays several voice messages to describe the state of the pillbox. The embodiment plays the day, date and time, the dose schedule, an adherence summary, and instructions for signing on to the patient web site. The second circular cutout 20 is for plugging in a 5V DC power cord. The embodiment uses a 2.4 A wall transformer supply (12 W) similar to that used to charge cell phones. The last circular cutout 21 on the back of the enclosure is for the password reset pushbutton. When this recessed button is pushed, it resets the web site usernames and passwords of the patient and the guardian to their default values. This pushbutton is flush with the back of the pillbox to avoid accidental activation. It can be pushed using a pen or a paper clip.

FIG. 2B shows the cutouts in the cover of the enclosure. There are four holes 22 for mounting the pill trays to the top of the enclosure. There are also two horizontal slots 23 to accommodate the ribbon cables that connect the instrumented pill tray PCBs to the measurement and control PCB inside the enclosure.

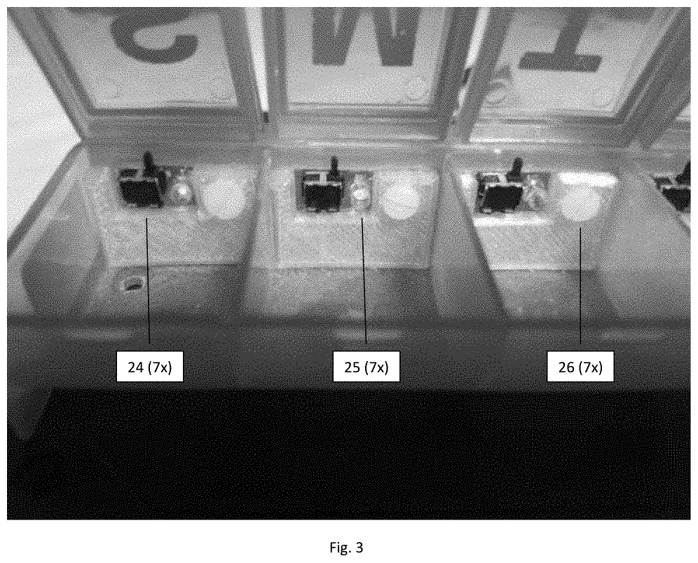

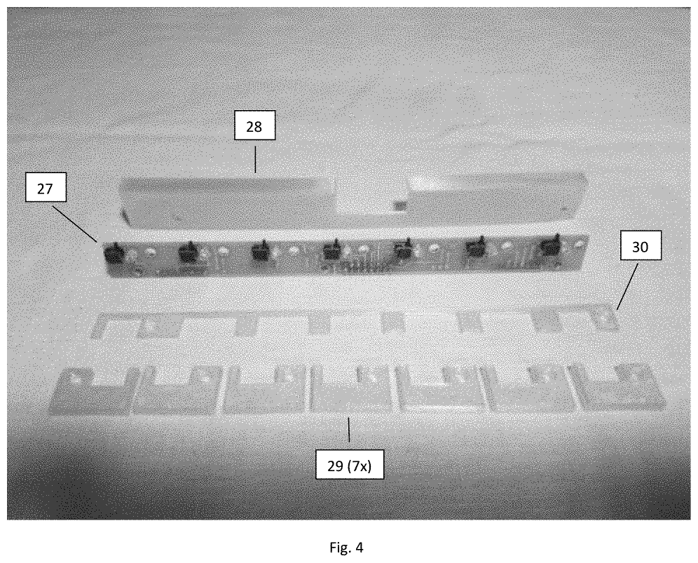

FIG. 3 shows a close up view of the weekly pill trays on the top of the pillbox. There are seven bins in each weekly pill tray and each bin contains a micro switch 24 for detecting the open/closed state of the bin lid, and a miniature bright white bin LED 25. The bin LED blinks when it is time to take a dose and it shows the patient which bin contains the current dose. The seven bracket mounting screws 26 secure the pill tray PCB to the back of the pill tray using the bin brackets 29 shown in more detail in FIG. 4.

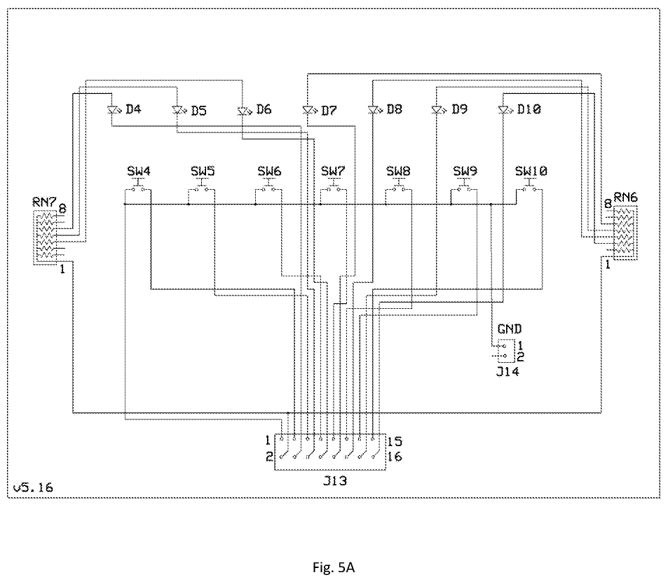

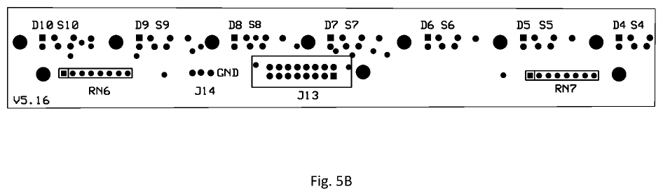

FIG. 4 shows a disassembled view of the pill tray PCB assembly that is attached to the back of each pill tray. The top row shows the pill tray cover 28. The pill tray cover is 3D printed using PLA filament. The second row is the pill tray PCB 27 that includes the seven micro switches and seven LEDs. They protrude through horizontal slots (not shown) in the back of each pill tray bin. The third row shows a custom designed gasket 30 that is 3D printed using PLA filament. The gasket thickness is less than 1 mm and is precisely controlled. This allows each micro switch to be precisely positioned in order to ensure reliable operation of the micro switches. In an alternative embodiment, one can manufacture new pill trays with covers specifically designed to accommodate the micro switches. The final row in FIG. 4 shows seven individual bin brackets 29 that are positioned inside the bins at the back. Bin brackets are used to fasten the pill tray PCB to the back of the pill tray with the pill tray gasket sandwiched between the front of the PCB and the back of the pill tray. The pill tray cover then goes over the exposed back of the PCB. A 16-conductor ribbon cable is used to connect each pill tray PCB to the measurement and control PCB inside the pillbox. A schematic diagram of the pill tray PCB is shown in FIG. 5A, and a pill tray parts layout diagram is shown in FIG. 5B.

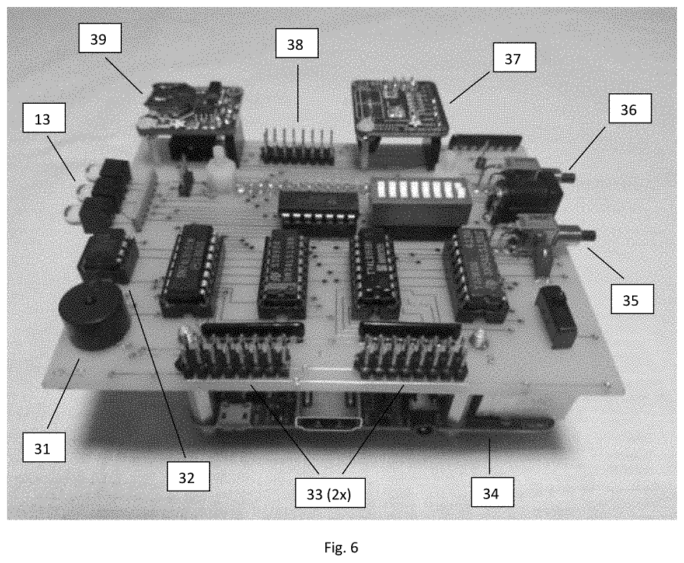

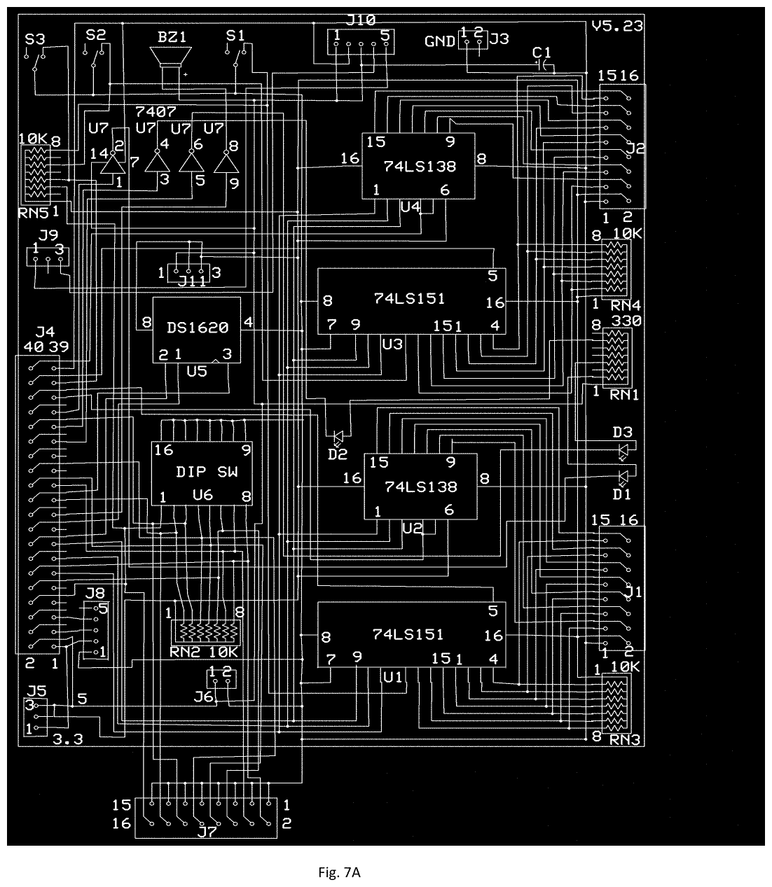

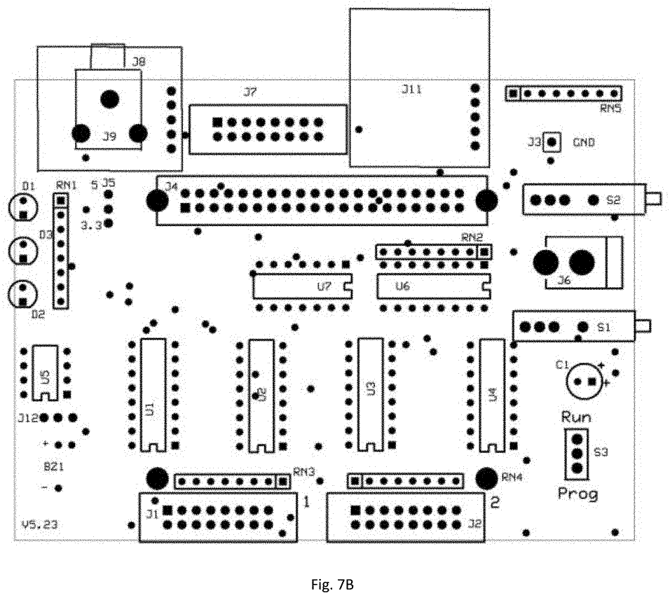

FIG. 6 shows the measurement and control PCB. This is a custom PCB that provides a hardware interface between the processor board 34 positioned below (Raspberry Pi 3B+) and the rest of the pillbox hardware. The measurement and control PCB connects directly to the processor board using its 40-pin GPIO buss. Numerous measurement functions are performed by the measurement and control PCB using multiplexing circuits. The embodiment monitors, in real time, 14 micro switches 24, two pushbuttons 35-36, eight external switches 38, one temperature sensor 32, and one real time clock 39. De-multiplexing circuits are used to implement numerous control functions including controlling 17 LEDs 13, 25 one sound actuator 31, and one audio amplifier 37. A schematic diagram of the measurement and control PCB is shown in FIG. 7A, and a measurement and control PCB parts layout diagram is shown in FIG. 7B.

Power Failures

The pillbox has been designed to respond gracefully in the event of a power failure. The pillbox can be unplugged at any time and then plugged back in. This is a “cold boot” of the system, and normal operation is restored once the power up sequence is complete. During a power outage, all patient dosage history data and patient adherence data are preserved along with the configuration parameters used to customize the pillbox for use by the patient. In the event of a prolonged power failure, there will be no reminders issued while power is out, but the patient can continue to take medicine. When power returns the state of the pillbox bins will have changed. Consequently, any doses taken during the power failure will be recorded when power returns. As an option, a rechargeable 5V battery pack similar to those used with cell phones can be used as a standby power source.

Dose Reminders and Types

Given the flexibility of the hardware design, the pillbox allows for numerous user features to facilitate patient adherence. To begin with, there are two classes of dose reminders provided, local reminders and remote reminders. Local reminders include light (blinking LEDs), sound (beeping), and descriptive voice messages. Local reminders are available for local doses which include pillbox doses and external doses. Recall that external doses are taken at home, but they do not fit in the pillbox bins so they are stored in their original medicine containers. Examples of external doses include items such as inhalers, eye drops, injections, liquid medication, and pills in blister packs. Each type of local reminder can be enabled or disabled. In the case of sound and voice, the spacing and the duration of the reminders can be adjusted.

There are also two types of remote reminders, email and text messages. Like the voice reminders, they are descriptive in the sense that they use information from the medication list and include the dose type, dose location, and the name or number of medications. Remote reminders are used for external doses and remote doses. Recall that a remote dose is a dose taken at work or away from home that is stored in a separate medicine container. Whereas pillbox doses are recorded when a bin is opened, external and remote doses are recorded when the patient replies to an email or text reminder. Remote doses also can be recorded using email and text commands or the patient web site.

Dose Frequencies

There is a dose frequency parameter that controls how often a dose is taken. The dose frequency ranges from 0 to 35. Zero selects a daily dose while 1 to 7 select a weekly dose where 1 selects Sunday, 2 selects Monday, and so on through 7 which selects a Saturday dose. Monthly doses are also possible using selections 8 to 35 where 8 selects day 1, 9 selects day 2, and so on through 35 which selects a dose on day 28. Note that since the same medicine can occur in more than one dose, this allows for doses whose medicine can be taken monthly, weekly, daily, or multiple times per day.

Remote Doses

When a patient is going to be away from home, there are a number of ways the pillbox can continue to be used. If the patient will only be gone part of a day, they can take their medicine with them and record the dose remotely using either email and text commands or the patient web site. When the patient will be away longer, they can take the pillbox with them. In this case, data lines one and two of the external memory file wifi.txt should be updated to reflect the Wi-Fi network at their new location. Still another possibility is to leave the pillbox home, but put it into something called the mode. This is done by using email and text commands or the patient web site to enter the start date and time and the end date and time of the vacation. When the start date and time arrives, all doses are automatically reclassified as remote doses, and when the end date and time arrives the process is reversed and the doses are restored to their original types. One advantage of the vacation mode is that local reminders are suppressed so individuals remaining at home are not bothered by light, sound, and voice reminders.

For remote doses there is a parameter called remote days that indicates which days of the week the dose is remote, otherwise the dose is external. Thus doses can be of one type on some days of the week and another type on other days of the week. For example, a remote days parameter of 0,1,1,1,1,1,0 indicates that the week days M-F are work days with remote doses, while the weekends are external doses taken at home.

Numerical Adherence Measures

A novel characteristic of the pillbox is that it provides simple numerical measures of patient adherence. Adherence measures are updated in real time every time a dose is taken. Each numerical measure ranges from 0% to 100% where 100% represents perfect adherence and 0% represents a complete lack of adherence. Arguably the most important adherence measure is dose quantity adherence. It measures the degree to which the patient takes the correct number of doses. For dose i let missed be the number of missed doses, extra be the number of extra doses, and days be the number of days in operation. Then the dose quantity adherence Aq for dose i is defined:

Aq=100*max{1−(missed+extra)/days,0}%

Note that as the quantity (missed+extra) goes from 0 to days, Aq decreases linearly from 100% to 0%. Thus if all the doses are taken, Aq=100%, if half are taken Aq=50%, and if none are take Aq=0%. The max or maximum function is included to make sure that Aq does not go negative if there are several extra doses taken.

Often it is important that doses be taken at the correct time. For example, some doses should be taken at meal time with food. Let Ti be the measured time for dose i, T be the scheduled time, and Tw be the radius of a user-selectable time window. Then the dose timing adherence At for dose i is defined:

At=100*max{1−mean(|Ti−T|)/Tw,0}%

Similar to dose quantity adherence, as the mean or average of |Ti−T] goes from 0 to Tw, At decreases linearly from 100% to 0%. For example, if mean(|Ti−T|)=Tw/2, then At=50%. Note that as the radius of the user-selectable time window, Tw, decreases the dose timing adherence becomes more sensitive to early and late doses.

Not only can dose timing be important, so can dose spacing. For example there can be some medications that interfere with other medicines so the spacing of doses must be carefully controlled. Let Si be the measured spacing between dose i and the previous dose, and let S be the scheduled spacing. Then the dose spacing adherence As for dose i is defined:

As=100*max{1−mean(|Si−S|)/S,0}%

Again as the mean or average of |Si−S| goes from 0 to S, As decreases linearly from 100% to 0%. If mean(|Si−S])=S/2, then As=50%. As a convenience to the user, a fourth numerical measure of patient adherence called overall adherence is computed. Overall adherence A is a weighted sum of dose quantity, dose timing, and dose spacing adherence defined as:

A=wq*Aq+wt*At+ws*As%

The adjustable weight coefficients are fractions between 0 and 1 that sum to unity, wq+wt+ws=1. Like the other measures of adherence, the overall adherence then ranges from 0% to 100%. Overall adherence is provided simply as a convenience to the user because it is not obvious what the weighting coefficients should be. However, it is clear that dose quantity adherence is the most important because if a dose is not taken then for that dose the dose timing and dose spacing are not defined. The default relative weights are wq=0.6, wt=0.2, and ws=0.2.

Minimum Dose Spacing

Another important aspect of dose spacing is governed by an adjustable parameter called minimum dose spacing. Over the counter pain medications often include instructions like take no more than two tablets per length of time. The embodiment of the pillbox is not a physically secure pill dispenser (with lockable lids) so it cannot guarantee minimum dose spacing by prohibiting patient access to the medication. However, it can guarantee that dose reminders follow a minimum dose spacing requirement. More specifically, suppose two doses are overdue. Once the first dose is taken email, text and voice messages are issued saying that reminders for the other dose are on hold until the minimum time between doses has elapsed. Whenever a new dose scheduled is submitted, the schedule will be rejected if it violates the minimum dose spacing requirement.

Direct Voice Messaging

Another feature of the pillbox is the implementation of something called direct voice messaging. Both the patient and the guardian have the ability to use the patient web site or email and text commands to schedule one or more brief messages. These voice messages are intended for the patient and they are played on their scheduled delivery dates when the patient opens a bin to take a pillbox dose. Examples of direct voice messages might include things like reminders of doctor's appointments, or even words of encouragement from the guardian. The messages are direct in the sense that with normal pillbox operation the patient is guaranteed to hear the message because it is played on the scheduled delivery date when the patient opens a bin to take a dose. This is in contrast, for example, to an answering machine where the patient must actively seek out the message and play it back in order to hear it.

Patient Environment

There is a feature of the pillbox that does not directly affect patient adherence. However, it does allow the guardian to monitor the well being of the patient. If the temperature of the patient's environment falls below a lower threshold or rises above an upper threshold, the guardian will receive email and text messages indicating that the patient's furnace or air conditioning are not operating properly. By using email and text commands or the patient web site the current temperature can be obtained and the lower and upper temperature limits can be adjusted.

The embodiment of the pillbox shown in FIG. 6 also includes a capability to monitor the open/closed states of up to eight external switches 38. Email and text message notices can be sent to the patient and the guardian whenever one of these switches changes state. Potential applications include such things as a switch to detect that the water level in a sump pump is high or perhaps a back door is open. In the embodiment the switches must be hard wired using a ribbon cable plugged into the external switch connector 38. However, in an alternative embodiment it is possible to instead implement a wireless connection to the external switches. Unlike the pillbox switches, the external switches do not measure patient adherence. Instead, like the temperature sensor, they remotely monitor the patient environment.

Remote Access

An important aspect of the operation of the pillbox is the ability to access the patient pillbox remotely from any location with a cell phone network or an Internet connection. Using remote access the patient can receive remote reminders and record remote doses. Equally important is the access provided to the guardian. With remote access, the guardian can easily monitor how well the patient is doing in adhering to the prescribed medicine regimen. Dosage history, adherence summaries, and patient data can be viewed on the patient web site, emailed to the guardian, and printed in hard copy form if desired. The guardian also can remotely reconfigure the operation of the pillbox at any time to better customize it so it matches the needs and desires of the patient.

There are two types of remote access supported. One is the through the patient web site and the other is with email and text commands. Access to the patient web site is both local on the patient's Wi-Fi network and remote over the Internet. For local access the patient must enter the private IP address of the pillbox in the address bar in the upper left corner of a web browser. The private IP address can be obtained by simply pressing the pillbox status pushbutton on the back of the pillbox. The private IP address is a static address that depends on the patient router, and is typically of the form 192.168.xxx.yyy. Here the third field xxx is between 0 and 255 and is the number of the subnetwork used by the router. The last field yyy is a user-selectable parameter called the pillbox id, an integer between 2 and 253. By using distinct pillbox ids, more than one pillbox can be accessed on the same local Wi-Fi network. The pillbox id parameter is listed in external memory file wifi.txt. It should be chosen to lie outside the range of IP addresses dynamically assigned by the patient router.

Remote access to the patient web site over the Internet is generally similar to local access, but with two important differences. First the patient router must be configured to use port forwarding. More specifically, port 80 which is the port used by web browsers must be forwarded to the private IP address of the pillbox. The details on how this is done depend on the make and model of the router. Port forwarding is normally set up when the pillbox is installed. A patient or guardian who is sufficiently familiar with computer hardware can set up port forwarding using the pillbox guide documentation on the patient web site. Once the router is configured for port forwarding, the only other thing that is needed is the public IP address of the patient's Wi-Fi network. This can be found by clicking on the Find icon on the patient web site. Alternatively, one can use a web browser on the patient Wi-Fi network and enter the search string whatismyip. The guardian then can access the login page of the patient web site by using the public IP address. In summary, remote access to the patient web site is just like local access but with the private IP address replaced by the public IP address.

Email and Text Commands

The second way to remotely access the pillbox is through a comprehensive set of email and text commands. This approach has a number of advantages. First, it does not require that the patient's router be configured for port forwarding. Second, it allows for remote access to more than one pillbox on the same local Wi-Fi network. Third, email and text commands can be sent using both a cell phone network and an Internet connection. For email and text commands to securely access a pillbox a number of requirements must be met. The command must be sent to the unique gmail address of the pillbox. In addition, the command must come from a limited set us users namely the patient, the guardian, or an administrator. Next, the email or text message must include the string <*serial_number*> where serial_number is the pillbox serial number typically listed on a sticker on the bottom of the pillbox. The serial number is a unique string of eight alpha-numeric characters. The final requirement is that the email or text message must contain one or more commands that follow the format [name: value] where name is the name of the command and value is the command value, an integer or a string. The embodiment of the pillbox includes 51 integer commands and 23 string commands that duplicate and extend the functionality of the patient web site. There are also a number of hidden administrator commands that are used for customer support and software maintenance. For example, there is an email command that allows the administrator to remotely update the pillbox software to a new version.

Patient Web Site

The patient web site provides a simple way to monitor how well the patient is adhering to the prescribed medicine regimen. It also provides a convenient way to configure the pillbox so it is optimized for use by the patient. The patient web site can be accessed locally on the patient's Wi-Fi network using the private IP address of the pillbox. Once the patient router has been configured to use port forwarding, the patient web site also can be accessed remotely over the Internet using the public IP address of the patient's Wi-Fi network. This document describes an embodiment of the patient web site. As experience with the pillbox accumulates, new features are added to the patient web site and existing features are improved in subsequent versions of the pillbox software.

Secure access to the patient web site starts with a login page that requires the user to supply a valid username and password. There are three levels of users, patient, guardian, and administrator. For the administrator the programming page also includes additional items such as pillbox diagnostics and software maintenance operations. These items are discussed in detail in the pillbox guide. The patient web site contains three pages in addition to the login page: dosage history, adherence summary, and programming.

Dosage History Page

A list of the items appearing on the dosage history page is shown in Table 2. The dosage history page starts with a set of links to the other pages and a link to the pillbox guide. The pillbox guide is a pdf document that describes the design and operation of the pillbox in some detail. The link section also includes date and time information, the pillbox private IP address, and the installed version of the pillbox software. Below the links section is a table of contents for the dosage history page with local links to the different items on the page

TABLE 2
Contents of Dosage History Page

| Item | Description |
|---|---|
| 1 | User Controls |
| 2 | Cumulative Patient Adherence |
| 3 | Dosage History |
| 4 | Bin Status |
| 5 | Pillbox Status |
| 6 | Email Data Files |
| 7 | Direct Voice Messaging |
| 8 | Scheduled Messages |
| 9 | Username and Password |

The first item in the contents is user controls. User controls allow the user to clear the patient data, restore the default parameters, restore the file wifi.txt, record a remote dose, select a week for the dosage history display, or find the public IP address of the patient's Wi-Fi network. All of the action labels on the left side of the user controls table are links to help tips that describe the item in more detail. This form of context-sensitive help is used throughout the patient web site. In order to activate a newly entered value for a user control item, the user must click on submit changes. Depending on the item selected, it may take some time before the action can be completed and reflected in a refreshed display of the page.

The next section of the dosage history page is a cumulative patient adherence table. It starts with how many days the pillbox has been in operation. There are separate lines for each dose. Each line starts with the dose number, scheduled dose time, dose frequency, dose type, and number pills in the dose. It then displays the measured adherence including dose quantity, dose timing, dose spacing, and overall adherence. The last row lists the averages of the adherence measures for the daily doses. Consequently, in the lower right corner there is a single numerical score measuring how well the patient is doing overall.

Below the cumulative patient adherence table is a dosage history display. By default, the most recent week of data is shown but earlier weeks can be selected using the user controls. The dosage history displays each day on a separate line labeled with the day and date. It then displays 24 hour slots where parentheses are used to show the scheduled times of pillbox doses and square brackets for external and remote doses. The dose number is plotted in the hour slot when the dose is taken. The total score at the end of the row indicates the percentage of scheduled doses that have been taken.

Next there is a small table that is two rows by seven columns. The rows are labeled with the bin or pill tray number and the columns are labeled with the days of the week. The bin status table is a real-time display showing which pill tray bins are open and which are closed.

Below the bin status table is a larger pillbox status table that includes several rows and four columns. The shaded yellow columns contain labels of the parameters whose values are listed in the white columns. The labels are links to help tips that describe the item in more detail. Most of the items in the pillbox status table are parameters whose values can be changed on the programming page.

The next table lists data files that can be emailed to the patient or the guardian. Some of the data files contain patient dosage history and adherence data while others are used to configure the pillbox for use by the patient. The email commands file is a listing of all email and text commands and their current values.

Below the data files table is a mechanism for scheduling direct voice messages to the patient. The currently scheduled voice messages are listed in a table along with their delivery dates. To insert a new message the user simply types it in and then enters a desired delivery date. Scheduled messages can be edited using delete, insert, and replace operations. Messages are sorted based on their delivery dates.

The last item on the dosage history page provides a means to change usernames and passwords. When the patient logs in to the web site, the patient username and password are shown and can be changed. Similarly, if the guardian is logs in, the guardian username and password can be updated. Recall that if the patient or the guardian forget or misplace their username or password, the recessed password reset pushbutton on the back of the pillbox can be used to restore the usernames and passwords to their default values. When this is done, the patient and guardian are sent emails informing them of the change.

Adherence Summary Page

A list of the items appearing on the adherence summary page is shown in Table 3. Like the dosage history page, the adherence summary page starts out with links to other pages followed by a table of contents with local links to the items on the page.

TABLE 3
Contents of Adherence Summary Page

| Item | Description |
|---|---|
| 1 | Medication List |
| 2 | Edit medication list |
| 3 | Dose Quantity Adherence |
| 4 | Dose Timing Adherence |
| 5 | Dose Spacing Adherence |
| 6 | Cumulative Overall Adherence |
| 7 | Dose Distribution |
| 8 | Display Data Files |

The current medication list is shown with each row corresponding to one entry in the list. When new medicine is inserted, the user must supply the medicine name and dosage, the number of pills per dose, and the dose number each separated by blank space. The next item in the contents is a mechanism for editing the list of medicines using insert, delete, and replace operations. This provides an easy way for a user to create and edit the external memory file medication_list.txt that is read during the pillbox startup sequence. Once a medication list is created or edited, the pillbox software automatically creates descriptive dose reminders and a pillbox refill form. This form, which can be displayed and printed, is helpful when it comes time to refill the pillbox bins. In addition the pillbox refill form serves as a medication list that the patient can bring to a doctor's appointment.

Following the medication list are four adherence summary tables for dose quantity, dose timing, dose spacing, and overall adherence. There are separate lines in the tables for each dose. Each line starts by listing the dose number, scheduled dose time, dose frequency, dose type, the ideal value of the measured quantity, and the number of pills in the dose. The dose quantity table then lists the average adherence, average variation from the ideal adherence, missed doses, extra doses, and cumulative adherence over the number of days in operation. The dose timing adherence table lists the average dose time, average variation from the scheduled time, and cumulative dose timing adherence. Similarly, the dose spacing table lists the average dose spacing, average variation from the scheduled spacing, and cumulative dose spacing adherence.

Next there is a large dose distribution table. This table shows when each dose was taken over the period of time the pillbox has been in operation or since the last time the patient data was cleared. As with the dosage history display, parentheses are used to show the scheduled times of pillbox doses, and square brackets are used to show external and remote doses. By carefully examining the dose distribution histogram table one can tell if the patient tends to take certain doses early or late and by how much.

The last table on the adherence summary page is a display data files table. Seven data files can be displayed and printed if desired. They include files containing dosage history and adherence data, configuration data, and email and text commands.

Programming Page

A list of the items appearing on the programming page is shown in Table 4. Whereas the previous two pages focus on presenting dosage history and adherence data, the programming page is designed to help the user configure the pillbox to fit the needs and desires of the patient. This page starts like the other pages with a links section and a local table of contents section.

TABLE 4
Contents of Programming Page

| Item | Description |
|---|---|
| 1 | Dose Schedule Settings |
| 2 | Temperature Settings |
| 3 | Vacation Mode Settings |
| 4 | Operating Mode Settings |
| 5 | Remote Operation Settings |
| 6 | Contact Information Settings |
| 7 | Pillbox Gmail Account Settings |
| 8 | Real Time Clock Settings |

The first item in the contents allows the user to configure the dose schedule. This includes strings that specify the dose times, dose types, dose frequencies, remote days, and adherence weights. The user also can select the minimum dose spacing, and the size of the time window. For more than one dose per day items within strings are separated by commas with no blank space.

Next there is a temperature settings table where the user can select both a low temperature limit and a high temperature limit. The safe zone for the patient environment lies between these limits. There is a third parameter called the temperature offset. This is used to calibrate the temperature sensor so it produces an accurate reading at room temperature. For proper calibration, one must wait until the pillbox has warmed up because there can be self heating effects from the pillbox electronics.

Following the temperature settings are vacation mode settings. Here the user enters strings that specify the start date and time and the end date and time of a vacation. To disable the vacation mode one simply sets the start and end dates to be earlier than the current date. Otherwise, once the current date and time reaches the start date and time, the pillbox will automatically enter the vacation mode (all doses remote), and will remain there until the end date and time is reached.

Next there is a general group of settings called operating mode settings. They start with light and sound reminders that can be enabled or disabled. The duration and spacing of the sound and voice reminders also can be adjusted. Voice type can be selected and the voice volume (for festival voice) can be adjusted. Periodic pillbox power resets can be scheduled and the rate at which email and text commands are polled can be adjusted.

The operating mode settings are followed by remote operation settings where there is one column for the patient and another for the guardian. The settings include emailing and texting pillbox alerts, dose reminders, and doses taken. In addition, weekly adherence summaries and monthly dosage histories can be emailed.

In order for remote communications to be activated, patient and guardian contact information must be supplied using the contact information settings. The information required for each individual includes first and last names, email address, cell phone number, and cell carrier.

The next table is the pillbox gmail account settings. Each pillbox should have its own gmail account separate and distinct from the patient and guardian email accounts. The settings include the gmail address and the gmail password. The gmail address is the address that is used when email and text commands are sent to the pillbox.

The last table on the programming page is the real time clock settings table. It starts with a control that selects the time zone. Next it includes a control to enable or disable the use of system time. Normally system time is left enabled. System time is synchronized by periodically contacting a national NTP time server to maintain a real time clock. When system time is disabled, control is transferred to a hardware real time clock with battery backup. This mode is used when the pillbox is operated in a stand-alone mode that does not use an Internet connection. There is also a control to set the date and the time of the hardware real time clock.

When an administrator logs in to the patient web site, there are additional programming page controls for customer support operations including pillbox diagnostics and software maintenance.

Pillbox Software

The embodiment of the pillbox uses a Raspberry Pi 3B+ processor board that runs the Linux operating system. The software resides on a 16 GB micro SD and is written in C, PCP and HTML. Linux makes liberal use of files that enable the different parts of the software to communicate with one another. The pillbox software uses the folders listed in Table 5.

TABLE 5
Pillbox Software Folders

| Folder | Description |
|---|---|
| /home/pi/hma | Main program folder |
| /home/pi/hma/lib | Library folder |
| /home/pi/hma/test | Test programs folder |
| /home/pi/bak | Backup and restore folder |
| /home/pi/bak/bak_lib | Backup library folder |
| /home/pi/dat | Data folder |
| /home/pi/profiles | User profiles folder |
| /home/pi/mail | Email folder |
| /media/usb | Usb folder |
| /media/usb/zipped | Zipped files folder |
| /media/usb/history | User history folder |
| /var/www/html | Web site folder |
| /var/www/html/tips | Help tips folder |

The Hma Folder

The main program folder named hma, short for Home Meds Adherence, contains the main program hma_main.c. Following an initialization section, the main program goes into a loop that runs at about 600 Hz where a number of items are polled including bin status, pushbutton status, web site parameter status, and email and text command status. The main program file includes support functions: get_version, check_minute, check_day, dose_active, and update_globals. Function get_version finds various version numbers during initialization, check_minute and check_day are called once a minute and once a day, respectively, to perform various tasks as needed, and dose_active determines which dose, if any, currently has reminders active or on hold. Function update_globals is called when communications are received in the form of email and text commands or changes to web site parameters.

The hma folder also contains three files called global_constants, global_parms, and global_vars that define constants, parameters, and variables used throughout the program. Global parameters are variables whose values remain constant unless they are explicitly changed using the external memory file wifi.txt, the patient web site, or email and text commands. Global variables are patient data variables whose values change with time as a result of routine patient activity. Global parameters and variables must be kept up to date at all times in the event of a loss of power. This is achieved by saving the parameters and variables in hma folder files hma_parm.dat and hma_var.dat, respectively, whenever a parameter or variable changes.

The Lib Folder

The library folder lib is a large folder that contains most of the C functions used in the pillbox software. The lib folder includes several library files and each of these files consists of a number of related functions that are grouped to achieve a particular type of task. The library files that make of up the lib folder are listed in Table 6.

TABLE 6
Library Files in /home/pi/hma/lib

| Library File | Functions |
|---|---|
| hma_adherence_lib.c | Compute and display patient adherence |
| hma_cmds_lib.c | Execute email and text commands |
| hma_controls_lib.c | Scan email for valid commands |
| hma_list_lib.c | Process direct voice messages, medication lists |
| hma_debug_lib.c | Debugging operations for software development |
| hma_device_lib.c | Low level functions, configure GPIO bus |
| hma_files_lib.c | File processing, loading and saving patient profile |
| hma_email_lib.c | Send email and text messages, read bin switches |
| hma_main_lib.c | Monitor pill trays, play status messages |
| hma_network_lib.c | Configure Wi-Fi network, maintain Internet connection |
| hma_sensor_lib.c | Monitor temperature sensor |
| hma_sound_lib.c | Generate sounds, play text to speech messages |
| hma_time_lib.c | Monitor system time and local real time clock |
| hma_usb_lib.c | Configure and provide access to external memory stick |
| hma_userblock_lib.c | Backup and transfer data, maintain passwords |

There are too many individual functions in the lib folder to itemize and discuss here. However, the lib folder does include header files with the .h extension for each of the library files listed in Table 6. These header files list function prototypes for all of the functions in the library file. The functions themselves start with comment lines that describe the function purpose, calling sequence, and calling arguments. For convenience there is an overall header file named hma_header.h in the hma folder that lists the prototypes of all of the C functions in the pillbox software.

Support Folders

There are several general support folders in Table 5. The test folder contains programs and functions for testing various aspects of the pillbox hardware and software. The principle test program is hma_test.c.

The bak and bak_lib folders contain programs and functions used for software development including backup, restore, and install operations. The program hma_backup.c can be used to save the current version of the pillbox software to an external memory zip file or restore a previous version from an external memory zip file. There is also a program called hma_install.c that installs or updates linux packages used by the pillbox software such as the apache web server and getmail.

The next three folders in Table 5 contain temporary files and back up files. The dat folder includes configuration files and data files that are created during normal operation of the pillbox. The profiles folder contains backup copies of the patient configuration files in external memory. Finally the mail file collects email messages sent to the pillbox gmail address.

There are three usb folders that are used. They are named usb folders because the embodiment of the pillbox uses a USB flash drive for the external memory stick. Folder usb contains the plain text configuration files wifi.txt and medication_list.text used to customize the pillbox for use by the patient. The zipped folder contains previously saved versions of the pillbox software, and the history folder contains the patient monthly dosage histories that have been accumulated to date.

The Html Folder

The last two folders in Table 5 are associated with the patient web site and contain files and functions written in PCP and HTML. Folder html contains files index.php for the login page, hma_dosage.php for the dosage history page, hma_adherence.php for the adherence summary page, and hma_prog.php for the programming page. The tips folder contains help tip files for the labels that appear throughout the patient web site.

There are two distinct parts of the pillbox software, the part written in C and the part written in PHP and HTML. These parts must communicate with one another to make the pillbox software fully operational. The lib folder function write_c2php.c starts by writing a dat folder file named c2php.dat that contains parameters and variables destined for the html folder. A companion html folder function named readc2php.php reads the contents of that data file. Whenever a web site parameter is changed by the user, html folder function write_php2c.php writes a second dat folder file named php2c.dat that contains the updated parameters. Main program hma_main.c detects the presence of this file during polling and reads its contents using the companion lib folder function read_php2c.c. Next the hma folder function update_globals is called by hma_main.c to process and save the new web site parameters. In this way the two parts of the pillbox software, written in different programming languages, communicate with one another through a pair of data files.

### CONCLUSIONS, RAMIFICATIONS, AND SCOPE

The smart pill organizer is a flexible device that facilitates patient adherence by remotely monitoring medicine usage in a home. It does so by supporting a variety of dose types including pillbox doses taken a home and stored in the pillbox bins, external doses also taken at home but stored in their original medicine containers, and remote doses taken at work or away from home and stored in separate medicine containers. A plurality of bidirectional communication paths are provided between the pillbox and a guardian who oversees the progress of the patient in adhering to a prescribed medicine regimen. Both the guardian and the patient can request patent data from the pillbox, and send commands to the pillbox to configure it to meet needs and desires of the patient. One avenue of communication is a locally hosted patient web site that is available both locally on the patient's Wi-Fi network and remotely over the Internet. Access to the pillbox is also available more directly through the use of a comprehensive set of email and text commands. This communication channel has the advantage that it can be activated through a cell phone network or an Internet connection. The pillbox provides direct numerical measures of patient adherence ranging from 0% to 100% that include dose quantity, dose timing, dose spacing, and overall adherence. For example, if only half of the doses are taken, but the ones that are taken are taken on time, then the dose quantity adherence is 50%, and the dose timing adherence is 100%.

The detailed description section discussed the first embodiment of the smart pillbox. Several alternative embodiments are possible. The embodiment shown in FIG. 1 allows for up to two pillbox doses per day and up to four total doses per day. An alternative embodiment based on a larger enclosure can readily accommodate up to four pill trays to support up to four pillbox doses per day. Furthermore the software can be updated to allow for a total of six or even eight total doses per day.

The first embodiment described the use of a Wi-Fi network to access the patient web site. It is also possible to use a wired network connection. Indeed, the back of the pillbox already has a wired network connector, and if it is used the pillbox automatically selects the wired network when it is powered up. Another possible embodiment uses no Internet service at all. This is possible because the pillbox already includes a real time clock with battery backup. Using a router, the patient still can access the web site locally to examine patient data and configure the pillbox. If no router is present, another way to configure the pillbox is through the use of the external memory stick. The first embodiment uses a USB flash drive for the external memory stick, but alternative embodiments can use any type of portable external memory device that is capable of storing files.

The first embodiment uses mechanical micro switches 24 as shown in FIG. 3 to monitor the state of the pill tray lids. Another embodiment can use alternative technologies such as optical sensors, proximity sensors, or capacitive sensors to detect access to the pill tray bins. The lids of the pill tray bins on the first embodiment are not lockable. In an alternative embodiment, pill trays can be manufactured with lids that can be locked or unlocked under software control thus turning the smart pillbox into a physically secure pill dispenser. An alternative embodiment based on custom manufactured pill trays also can remove the need for the switch-positioning gasket 30 shown in FIG. 4.

The first embodiment includes a external switch connector 38 in FIG. 6 that supports up to eight external switches that can be placed throughout the home. The external switches of the first embodiment must be hard wired directly to the ribbon cable. In an alternative embodiment the ribbon cable can be terminated with an optional Wi-Fi transceiver so the external switches can communicate wirelessly with the pillbox. This will simplify the placement of the external switches.

The alternative embodiments summarized above should not be construed as an exhaustive list that limits the scope of embodiments; instead they provide illustrations of several of the embodiments that are possible.

### REFERENCES

[1] Vermeire, H. Hernshaw, P. Van Royen, and J. Denekens, “Patient Adherence to treatment: three decades of research. A comprehensive review. J. of Clinical Pharmacy and Therapeutics, Vol. 26, pp. 331-342, 2001.
[2] D. L. Sackett, R. B. Haynes, E. S. Gibson, et al. “Randomized clinical trial of strategies for improving medication compliance in primary hypertension,” Lancet, Vol. 1, pp. 1205-1207, 1975.
[3] J. L. Donovan, D. R. Blake, “Patient non-compliance: deviance or reasoned decision-making?” Social Science and Medicine, Vol. 34, pp. 507-=513, 1992.
[4] L. S. Morris, R. M. Schulz, “Patient compliance: an overview.” J. of Clinical Pharmacy and Therapeutics, Vol. 17, pp. 183-195, 1992.
[5] J. L. Donovan, “Patient decision making. The missing ingredient in compliance research”, Int. J. of Technology Assessment in Health Care, Vol. 11, pp. 443-445, 1995.
[6] S. Griffith, “A review of the factors associated with compliance and the taking of prescribed medicines,” British J. of General Practice, Vol. 40, pp. 114-116, 1990.
[7] S. A. Vik, and C. J. Maxwell, and D. B. Hogan, “Measurement, correlates, and health outcomes of medication adherence among seniors,” Annals of Pharmacotherapy, Vol. 38, pp. 303-312, 2004.
[8] L. Gordis, “Conceptual and methodologic problems in measuring patient compliance,” Compliance in Health Care, B. Haynes, D. W. Taylor, and D. W. Sackett, Editors, Johns Hopkins University Press: pp. 23-45, 1979.
[9] Stewart} M. A. Stewart, “The validity of an interview to assess patients' drug taking,” American J. of Preventative Medicine, Vol. 3, pp. 95-100, 1987.
[10] J. A. Cramer, “Microelectronic systems for monitoring and enhancing patient compliance with medication regiments,” Drugs, Vol. 49, pp. 321-327, 1995.
[11] H. L. Figge, “Electronic tools to measure and enhance medication adherence”, US Pharmacist., Vol. 36(4), pp. 6-10.
[12] The MedMinder Pill Dispenser, www.medminder.com
[13] T. Charles, D. Quinn, M. Weatherall, et al. “An audiovisual reminder function improves adherence with inhaled corticosteroid therapy in asthma,” J. Allergy Clin. Immunol. Vol 119, pp. 811-816, 2007.
[14] Seven Day XL Contour Pill Reminder—Extra Large from Pill Thing, www.pillthing.com
[15] DC-47P Heavy-Duty Electronics Enclosure, www.polycase.com

What is claimed is:

1. A smart pill organizer to facilitate patient adherence by remotely monitoring medicine usage in a home, comprising: a plurality of instrumented pill trays each with a plurality of bins having curved bottoms and hinged lids, mounted on top of an enclosure that includes status lights on the front and pushbuttons, an external memory stick, a network connector and a power connector on the back, custom electronics inside for performing real time measurement and control operations, a processor inside that functions as a local server that hosts a patient web site, and processor software that implements functionality comprising:
(a) a plurality of means of securely communicating patient data from the smart pill organizer to a guardian who remotely monitors patient activity,
(b) a plurality of means of securely communicating commands from the guardian to the smart pill organizer to configure the smart pill organizer for use by a patient, and
(c) a plurality of reminders, alerts, and dose types where a dose is any collection of medications that can be taken at the same time.

2. The smart pill organizer of claim 1 wherein the plurality of means of securely communicating patient data from the smart organizer to the guardian in (a) comprise the patient web site and a set of email and text message commands.

3. The smart pill organizer of claim 1 wherein the plurality of means of securely communicating commands from the guardian to the smart pill organizer in (b) comprise the patient web site and a set of email and text message commands.

4. The smart pill organizer of claim 1 wherein the plurality of reminders in (c) comprise light reminders, sound reminders, and descriptive voice, email and text reminders, or a combination thereof.

5. The smart pill organizer of claim 1, wherein the plurality of reminders in (c) comprise dose reminders that remain on hold until sufficient time has elapsed to satisfy an adjustable minimum dose spacing requirement.

6. The smart pill organizer of claim 1 wherein each smart pill organizer bin includes a light source that activates to inform the patient which bin to remove medicine from.

7. The smart pill organizer of claim 1 wherein the plurality of alerts in (c) comprise alerts for a missed dose, an extra dose, doses too close together, a dose from the wrong bin, a dose from the wrong day, a bin already open, pillbox empty, or a combination thereof.

8. The smart pill organizer of claim 1 wherein the plurality of dose types in (c) comprise doses whose medicine is taken monthly, weekly, daily, multiple times per day, or a combination thereof.

9. The smart pill organizer of claim 1 wherein the plurality of dose types in (c) comprise pillbox doses taken at home and stored in the smart pill organizer bins, external doses taken at home and stored in their original medicine containers, remote doses taken at work or away from home and stored in separate medicine containers, or a combination thereof.

10. The smart pill organizer of claim 1 wherein the plurality of dose types in (c) comprise doses that can be of one type on some days of the week and another type on other days of the week.

11. The smart pill organizer of claim 1 wherein the pushbuttons comprise a button that resets the patient and guardian usernames and passwords to their default values, a button that plays a status message that includes a patient adherence summary, or a combination thereof.

12. The smart pill organizer of claim 1, wherein local access to the patient web site from the patient's Wi-Fi network is available both with and without an Internet connection.

13. The smart pill organizer of claim 1, wherein remote access to the smart pill organizer is available using both a cell phone network and an Internet connection.

14. The smart pill organizer of claim 1, further comprising a plurality of means of recording doses including opening a smart pill organizer bin, accessing a patient web site, replying to an email reminder, replying to a text reminder, sending an email command, sending a text command, or a combination thereof.

15. The smart pill organizer of claim 1, further comprising a plurality of means of securely communicating with the smart pill organizer to send direct voice messages played for the patient on scheduled delivery dates when the patient opens a smart pill organizer bin.

16. The smart pill organizer of claim 1, further comprising a plurality of means of remotely monitoring the state of the patient's environment comprising the temperature, a plurality of external switches throughout a home, or a combination thereof.

17. The smart pill organizer of claim 1, further comprising a plurality of means of creating and editing a patient medication list for doctor's appointments that generates descriptive dose reminders and a smart pill organizer refill form.

18. The smart pill organizer of claim 1, further comprising a means of securely communicating commands from an administrator to the smart pill organizer to provide customer support and software updates.

19. The smart pill organizer of claim 1, further comprising a means of securely communicating with a plurality of smart pill organizers that are connected to the same local Wi-Fi network.

20. The smart pill organizer of claim 1, wherein:
(I) the plurality of means of securely communicating patient data from the smart pill organizer to the guardian in (a) comprises a patient web site and a set of email and text message commands,
(II) the plurality of means of securely communicating commands from the guardian to the smart pill organizer in (b) comprises a patient web site and a set of email and text message commands,
(III) the plurality of reminders in (c) comprise:
(i) light reminders, sound reminders, and descriptive voice, email and text reminders, or a combination thereof,
(ii) dose reminders that remain on hold until sufficient time has elapsed to satisfy an adjustable minimum dose space requirement, and
(iii) a light source for each smart pill organizer bin that activates to inform the patient which bin to remove medicine from,
(IV) the plurality of alerts in (c) comprises alerts for a missed dose, an extra dose, doses to close together, a dose from the wrong bin, a dose from the wrong day, a bin already open, pillbox empty, or a combination thereof,
(V) the plurality of dose types in (c) comprise:
(i) doses whose medicine is taken monthly, weekly, daily, multiple times per day, or a combination thereof,
(ii) pillbox doses taken at home and stored in the smart pill organizer bins, external doses taken at home and stored in their original medicine containers, remote doses taken at work or away from home and stored in separate medicine containers, or a combination thereof, and
(iii) doses that can be of one type on some days of the week and another type on other days of the week,
(VI) the pushbuttons comprise a button that resets the patient and guardian usernames and passwords to their default values, a button the plays a status message that includes a patient adherence summary, or a combination thereof,
(VII) local access to the patient web site from the patient's Wi-Fi network is available both with and without an Internet connection,
(VIII) remote access to the smart pill organizer is available using both a cell phone network and an Internet connection,
and where the smart pill organizer further comprises: a plurality of means of recording doses comprising opening a smart pill organizer bin, accessing a web site, replying to an email reminder, replying to a text reminder, sending an email command, sending a text command, or a combination thereof,
(d) a plurality of means of securely communicating with the smart pill organizer to send direct voice messages played for the patient on scheduled delivery dates when the patient opens a smart pill organizer bin,
(e) a means of remotely monitoring the state of the patient's environment comprising the temperature, a plurality of external switches throughout the home, or a combination thereof,
(f) a plurality of means of creating and editing a patient medication list for doctor's appointments that generates descriptive dose reminders and a smart pill organizer refill form,
(g) a means of securely communicating email commands from an administrator to the smart pill organizer to provide customer support and software updates, and
(h) a means of securely communicating with a plurality of smart pill organizers connected to the same local Wi-Fi network.
